(19) United States
(12) Patent Application Publication
(10) Pub. No.: US 2007/0082385 A1
(43) Pub. Date: Apr. 12, 2007

(54) Fermentation process

(75) Inventors: Mads Torry Smith, Raleigh, NC (US); Gregory Alan Crabb, Wake Forest, NC (US); John Francis Ress, Youngsville, NC (US)

Correspondence Address: NOVOZYMES NORTH AMERICA, INC., 500 FIFTH AVENUE, SUITE 1600, NEW YORK, NY, 10110, US

(73) Assignee: NOVOZYMES NORTH AMERICA, INC., FRANKLINTON, NC (US)

(21) Appl. No.: 10/579,403

(22) PCT Filed: Dec. 10, 2004
(86) PCT No.: PCT/US04/41969

§ 371 Date: May 12, 2006

Related U.S. Application Data
(60) Provisional application No. 60/577,773, filed on Jun. 8, 2004.
(60) Provisional application No. 60/529,202, filed on Dec. 12, 2003.

(51) Int. Cl. C12P 7/06

(52) U.S. Cl. 435/161

(57) ABSTRACT

The present invention provides a process of producing a fermentation product including introducing the fermenting organism into the fermentation medium and thereafter adding a carbohydrate generating enzyme after the lag phase of the fermenting organism and then carry out fermentation under conditions suitable for producing the fermentation product in question. The invention also relates to fermentation product producing processes including the fermentation process of the invention.

### FIELD OF THE INVENTION

The present invention relates to enzymatic processes for producing fermentation products, wherein the fermenting organism performance during fermentation is improved and the yield is increased.

### BACKGROUND OF THE INVENTION

Fermentation processes are used for making a vast number of commercial products, including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds which are difficult to produce synthetically. Fermentation processes are also commonly used in the consumable alcohol (e.g., beer and wine), dairy (e.g., in the production of yogurt and cheese), leather, and tobacco industries.

There is a need for further improvement of fermentation processes and for improving processes that include a fermentation step.

### SUMMARY OF THE INVENTION

The present invention provides improved processes for producing a fermentation product. The fermenting organism performance, such as yeast performance, during fermentation is improved resulting in an increased fermentation product yield. The present invention also provides improved processes for producing ethanol using one or more of the processes described herein.

The term “increased yield” means that the product yield provided by a process of the invention is higher compared to the yield of a corresponding process under the same conditions wherein the carbohydrate-source generating enzyme(s) is(are) added during the propagation of the fermenting organisms.

The term “lag phase” means the period preceding the exponential growth phase when cells may be metabolizing but are not yet growing

The term “growth” means an increase in cell number.

The term “exponential growth” means growth of a microorganism where the cell number doubles within a fixed time period.

Thus, in the first aspect the invention relates to a process of producing a fermentation product in a fermentation medium which process comprises a fermentation step, which fermentation step includes subjecting liquefied mash to a carbohydrate-source generating enzyme and a fermenting organism, wherein the process comprises:

i) introducing the fermenting organism into the fermentation medium, ii) adding said carbohydrate-source generating enzyme after the lag phase of the fermenting organism,

iii) fermenting under conditions suitable for producing the fermentation product.

In a second aspect the invention relates to a process for producing a fermentation product, especially ethanol, comprising

(a) milling whole grains;

(b) liquefying the product of step (a);

(c) introducing the fermenting organism into the liquefied product obtained in step (b),

(d) adding the carbohydrate-source generating enzyme after the lag phase of the fermenting organism, and

(e) fermenting under conditions suitable for producing the desired fermentation product, especially ethanol.

Other fermentation products, e.g., listed in the “Background”-section herein may also be produced this way.

### DETAILED DESCRIPTION OF THE INVENTION

The present invention provides improved processes for producing a fermentation product. The fermenting organism, including yeast, performance during fermentation is improved resulting in an increased fermentation product yield. The present invention also provides improved processes for producing ethanol and other fermentation products using one or more of the processes described herein.

Fermentation Processes of the invention

In the beginning of a SSF batch fermentation a lag phase is introduced when the batch is inoculated with a fermenting organism, such as yeast. When a carbohydrate-source generating enzyme, such as glucoamylase, is dosed simultaneously with the yeast, buildup of substrate to high concentrations occurs. The initially high sugar level induces stress which can possibly lead to increased glycerol formation (fermenting organism/yeast stress response). The extra substrate used for the glycerol production is then lost for, e.g., ethanol formation.

The inventors have found that delayed addition of a carbohydrate-source generating enzyme to liquefied mash increases the product yield of a fermentation process. More specifically the inventors have shown that delayed addition of glucoamylase to a corn mash fermentation results in an increased ethanol yield.

Without being limited to any theory the increase in the fermentation product yield observed is believed to be due to the fermenting organism being allowed time to acclimatize initially before being subjected to a rapid sugar release resulting from addition of glucoamylase or another carbohydrate-source generating enzyme. In other words, inhibition of the fermenting organism due to a substrate “shock” is eliminated or at least minimized. The fermenting organism exit the initial lag phase before being subjected to high sugar levels.

Specifically the inventors found that the ethanol yield in a 65 hours corn mash batch fermentation was increased with a 3.5 hours delayed in addition of glucoamylase.

Another reason for the increase in production yield could be that the carbohydrate concentration available relative to the carbohydrate source demand of the fermenting organism is optimized by delaying the addition of the carbohydrate-source generating enzyme.

In general, tailing is observed at the end of fermentation, so a delay in enzyme addition does not alter the fermentation time significantly. In a standard fermentation on corn mash wherein enzymes and yeast are added simultaneously at the onset of the fermentation, a sugar peak higher than 150 g glucose/liter may be observed. The delayed enzyme addition provides a higher yield at a standard enzyme dose.

Thus, in the first aspect, the invention relates to a process of producing a fermentation product in a fermentation medium which process comprises a fermentation step, which fermentation step includes subjecting liquefied mash to a carbohydrate-source generating enzyme and a fermenting microorganism, wherein the process comprises:

i) introducing the fermenting organism into the fermentation medium,

ii) adding said carbohydrate-source generating enzyme after the lag phase of the fermenting organism.

In one embodiment the carbohydrate-source generating enzyme is added when the exponential growth phase of the fermenting organism is initiated.

Fermentation

“Fermentation” refers to any fermentation method or process comprising a fermentation step. A fermentation process of the invention includes, without limitation, fermentation methods or processes used to produce alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. Fermentation processes also include fermentation processes used in the consumable alcohol industry (e.g., beer and wine), dairy industry (e.g., fermented dairy products), leather industry and tobacco industry. Preferred fermentation processes include alcohol fermentation processes, as are well known in the art. Preferred fermentation processes are anaerobic fermentation processes, as are well known in the art.

In a preferred embodiment, the process of the present invention is used in combination with a saccharification process, in which additional enzymatic activities, such as esterase, such as lipase and/or cutinase, phytase, laccase, cellulase, xylanase, alpha-amylase, glucoamylase, or mixtures thereof, may be used in processing the substrate, e.g., a starch substrate.

In yet another preferred embodiment, the process of the invention is used in the production of ethanol.

Fermentation Media

“Fermentation media” or “fermentation medium” refers to the environment in which the fermentation is carried out and which includes the fermentation substrate, that is, the carbohydrate source that is metabolized by the fermenting organism(s). The fermentation media, including fermentation substrate and other raw materials used in the fermentation process of the invention may be processed, e.g., by milling and liquefaction or other desired processes prior to the fermentation. Accordingly, the fermentation medium can refer to the medium before or after the fermenting organism(s) is(are) added, such as, the medium in or resulting from a liquefaction step, as well as the media which comprises the fermenting organisms, such as, the media used in a simultaneous saccharification and fermentation process (SSF).

Fermenting Organism

“Fermenting organism” refers to any organism, including bacterial and fungal organisms, suitable for use in a desired fermentation process. Especially suitable fermenting organisms according to the invention are able to ferment, i.e., convert, sugars, such as glucose or maltose, directly or indirectly into the desired fermentation product. Examples of fermenting organisms include fungal organisms, such as yeast. Preferred yeast includes strains of the Saccharomyces spp., and in particular, Saccharomyces cerevisiae. Commercially available yeast include, e.g., Red Star®/Lesaffre Ethanol Red (available from Red Star/Lesaffre, USA) FALI (available from Fleischmann's Yeast, a division of Burns Philip Food Inc., USA), SUPERSTART (available from Alltech), GERT STRAND (available from Gert Strand AB, Sweden) and FERMIOL (available from DSM Specialties).

Fermentation Substrate

Any suitable substrate or raw material may be used according to the present invention. The substrate is generally selected based on the desired fermentation product and the process employed, as is well known in the art. Examples of substrates suitable for use in the processes of present invention, include starch-containing materials, such as tubers, roots, whole grains, corns, cobs, wheat, barley, rye, milo or cereals, sugar-containing raw materials, such as molasses, fruit materials, sugar, cane or sugar beet, potatoes, and cellulose-containing materials, such as wood or plant residues. The term “liquefied mash” includes any of the above raw materials, which have been subjected to liquefaction using any method known in the art. Preferred is enzymatically liquefied mash, especially liquefied corn mash.

Carbohydrate-Source Generating Enzymes

The term “carbohydrate-source generating enzymes” includes glucoamylases (being a glucose generators), and beta-amylases and maltogenic amylases (being maltose generators). Other enzymes producing other carbohydrates suitable for the fermenting organism in question is also contemplated according to the invention. A carbohydrate-source generating enzyme is capable of providing energy to the fermenting organism(s) used in the process of the invention and/or may convert the carbohydrate in question directly or indirectly to the desired fermentation product, preferably ethanol. The carbohydrate-source generating enzyme may be mixtures of enzymes falling within the definition. Especially contemplated mixtures are mixtures of at least a glucoamylase and an alpha-amylase, especially an acid amylase, even more preferred a fungal acid alpha-amylase. The ratio between fungal acid alpha-amylase activity (AFAU) per glucoamylase activity (AGU) (AFAU per AGU) may in one embodiment of the invention be at least 0.1, in particular at least 0.16, such as in the range from 0.12 to 0.50.

Examples of contemplated glucoamylases, alpha-amylases and beta-amylases are set forth in the sections below.

It is to be understood that the enzymes used according to the invention should be added in effective amounts.

Glucoamylase

A glucoamylase used according to the invention may be derived from any suitable source, e.g., derived from a micro-organism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381, WO 00/04136 add WO 01/04273 (from Novozymes, Denmark); the A. awamor glucoamylase (WO 84/02921), A. oryzae (Agric. Biol. Chem. (1991), 55 (4), p. 941-949), or variants or fragments thereof.

Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Corticium rolfsii glucoamylase (U.S. Pat. No. 4,727,046), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thennoamylolyticum (EP 135,138), and C. thermohydrosulfuncum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS (Dry Solids), preferably 0.1-10 AGU/g DS, such as about 2 AGU/g DS.

Amylase

According to the invention preferred alpha-amylases are of fungal or bacterial origin.

More preferably, the alpha-amylase is a Bacillus alpha-amylase, such as, derived from a strain of B. licheniformis, B. amyloliquefaciens, B. stearothermophilus, and B. subtilis. Other alpha-amylases include alpha-amylase derived from a strain of Bacillus sp., including Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. Examples of alpha-amylase variants and hybrids are described in WO 96/23874, WO 97/41213, and WO 99/19467. Other alpha-amylases include alpha-amylases derived from a strain of Aspergillus, such as, Aspergillus oryzae and Aspergillus niger alpha-amylases. In a preferred embodiment the alpha-amylase is an acid alpha-amylase. In a more preferred embodiment the acid alpha-amylase is a fungal acid alpha-amylase or a bacterial acid alpha-amylase. More preferably, the acid alpha-amylase is a fungal acid alpha-amylase derived from the genus Aspergillus. A commercially available acid fungal amylase is SP288 (available from Novozymes A/S, Denmark).

In a preferred embodiment, the alpha-amylase is an acid alpha-amylase. The term “acid alpha-amylase” means an alpha-amylase (E.C. 3.2.1.1) which added in an effective amount has optimum activity at a pH in the range of 3.0 to 7.0, preferably from 3.5 to 6.0, or more preferably from 4.0-5.0.

A preferred fungal acid alpha-amylase is a Fungamyl-like alpha-amylase. In the present disclosure, the term “Fungamyl-like alpha-amylase” indicates an alpha-amylase which exhibits a high homology (identity), i.e. more than 50%, 55%, 60%, 65%, 70%, 75%, 80%, 85% 90%, 95%, 96%, 97%, 98%, or even 99%% homology (identity) to the amino acid sequence shown in SEQ ID NO: 10 in WO 96/23874. When used as a maltose generating enzyme fungal alpha-amylases may be added in an amount of 0.001-1.0 AFAU/g DS, preferably from 0.002-0.5 AFAU/g DS, preferably 0.02-0.1 AFAU/g DS.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variant of set acid fungal amylase having at least 70% identity, such as at least 80% or even at least 90%, 95%, 96%, 97%, 98%, or 99% identity thereto is contemplated.

A preferred acid alpha-amylase for use in the present invention may be derived from a strain of B. Iicheniformis, B. amyloliquefaciens, and B. stearothernophilus.

Preferred commercial compositions comprising alpha-amylase include MYCOLASE from DSM (Gist Brocades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEYME FRED, SPEZYME™ AA, and SPEZYME™ DELTA AA (Genencor lnt.), and the acid fungal alpha-amylase sold under the trade name SP 288 (available from Novozymes A/S, Denmark). The alpha-amylase may be added in an amount that is well-known in the art. When measured in AAU units the acid alpha-amylase activity is preferably present in an amount of 5-50,0000 AAU/kg of DS, in an amount of 500-50,000 AAU/kg of DS, more preferably in an amount of 100-10,000 MU/kg of DS, such as 500-1,000 MU/kg DS. Fungal acid alpha-amylase are preferably added in an amount of 10-10,000 AFAU/kg of DS, in an amount of 500-2,500 AFAU/kg of DS, more preferably in an amount of 100-1,000 AFAU/kg of DS, such as approximately 500 AFAU/kg DS.

Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S. Maltogenic alpha-amylases are described in EP patent no. 120,693, U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference.

Beta-amylase

At least according to the invention a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is SPEZYME™ BBA 1500 from Genencor Int., USA.

In an embodiment, the fermentation process of the present invention is carried out as a simultaneous saccharification and fermentation step (SSF). In a preferred embodiment, the fermentation process is used for producing an alcohol, preferably ethanol. The presence of at least one carbohydrate-source generating enzyme may be used to raise the fermentation product yield, especially ethanol yield. In a preferred embodiment the fermentation is performed in the presence of one or more additional enzyme activities. The additional enzyme(s) may be introduced prior to, during/simultaneous with or after addition of the carbohydrate-source generating enzyme. Additional enzyme activities contemplated are esterase, such as lipase, phospholipase and/or cutinase, phytase, laccase, protease, cellulase, cellobiase, and, or a mixture thereof.

Growth Stimulators

In an embodiment of the process of the invention one or more growth stimulators are added to further improve the fermentation, and in particular, the performance of the fermenting organism, such as, rate enhancement and product yield. Preferred stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, e.g., Alfenore et al., Improving ethanol production and viability of Saccharomyces cerevisia by a vitamin feeding strategy during fed-batch process,” Springer-Verlag (2002), which is hereby incorporated by reference. Examples of minerals include minerals and mineral salts that can supply nutrients comprising P, K, Mg, S, Ca, Fe, Zn, Mn, and Cu.

Production of Enzymes

As disclosed above, the enzymes may be derived or obtained from any origin, including, bacterial, fungal, yeast or mammalian origin. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g. by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In a preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used in the present invention may be in any form suitable for use in the processes described herein, such as e.g. in the form of a dry powder or granulate, a non-dusting granulate, a liquid, a stabilized liquid, or a protected enzyme. Granulates may be produced, e.g. as disclosed in U.S. Pat. No. 4,106,991 and U.S. Pat. No. 4,661,452, and may optionally be coated by methods known in the art. Liquid enzyme preparations may, for instance, be stabilized by adding stabilizers such as a sugar, a sugar alcohol or another polyol, lactic acid or another organic acid according to established methods. Protected enzymes may be prepared according to the method disclosed in EP 238,216.

Production Processes

A preferred application of the fermentation process of the invention described herein is in an ethanol production process (e.g., for use as a fuel or fuel additive). The processes described herein can be used to increase the rate and/or yield of ethanol production.

Thus, in the second aspect the invention relates to a process for producing a fermentation product, especially ethanol, comprising

(a) milling whole grains;

(b) liquefying the product of step (a);

(c) introducing the fermenting organism into the liquefied product obtained in step (b),

(d) adding the carbohydrate-source generating enzyme after the lag phase of the fermenting organism.

(e) fermenting under conditions suitable for producing the fermentation product in question, especially ethanol.

In one embodiment the carbohydrate-source generating enzyme is added when the exponential growth phase of the fermenting organism is initiated.

Milling

In the production of ethanol and other starch-based fermentation products according to the invention, the raw material, such as whole grain, preferably corn, is milled in order to open up the structure and allow for further processing. Two processes are preferred according to the invention: wet milling and dry milling. Most used for ethanol production is dry milling where the whole kernel is milled and used in the remaining part of the process. Wet milling may also be used and gives a good separation of germ and meal (starch granules and protein) and is with a few exceptions applied at locations where there is a parallel production of syrups. Both wet and dry milling processes are well known in the art.

Liquefaction

Fermentation product production processes, such as ethanol production processes, generally involves the steps of liquefaction, saccharification, fermentation and optionally distillation. In the liquefaction process, milled gelatinized, e.g., (whole) grain raw material is broken down (hydrolyzed) into maltodextrins (dextrins). The hydrolysis may be carried out by acid treatment or enzymatically by alpha-amylase treatment, in particular with Bacillus alpha-amylases as will be described further below. Acid hydrolysis is used on a limited basis. The raw material is in one preferred embodiment of a process of the invention milled whole grain. However, a side stream from starch processing may also be used.

Any suitable liquefaction processes known in the art may also be used. Liquefaction is often carried out as a three-step hot slurry process. The slurry is heated to between 60-95° C., preferably 80-85° C., and the enzymes are added to initiate liquefaction (thinning). Then the slurry is jet-cooked at a temperature between 95-140° C., preferably 105-125° C. to complete gelatinization of the slurry. Then the slurry is cooled to 60-95° C. and more enzyme(s) is(are) added to finalize hydrolysis (secondary liquefaction). The liquefaction process is usually carried out at pH 4.5-6.5, in particular at a pH between 5 and 6. Milled and liquefied whole grains are known as mash.

The liquefaction process may be performed in the presence of an alpha-amylase. Preferred alpha-amylases are of fungal or bacterial origin. Bacillus alpha-amylases, variant and hybrids thereof, are specifically contemplated according to the invention. Well-known alpha-amylases include alpha-amylase derived from a strain of B. lichenifornis (commercially available as TERMAMYL™ from Novozymes A/S, Denmark), B. amyloliquefaciens, and B. stearothermophilus alpha-amylase (BSG). Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. Other alpha-amylase variants and hybrids are described in WO 96/23874, WO 97/41213, and WO 99/19467. Alpha-amylases derived from a strain of Aspergiltus include Aspergillus oryzae and Aspergillus niger alpha-amylases. Commercial alpha-amylase products and products containing alpha-amylases include TERMAMYL™ SC, FUNGAMYL™, LIQUOZYME™ and SAN™ SUPER.

Fungal alpha-amylases may be added in an amount of 0.001-1.0 AFAU/g DS, preferably from 0.002-0.5 AFAU/g DS, preferably 0.02-0.1 AFAU/g DS. Bacillus alpha-amylases may be added in effective amounts well known to the person skilled in the art.

Saccharification and Fermentation

To produce low molecular sugars DP₁₋₃ (i.e., carbohydrate source) that can be metabolized by a fermenting organism, such as, yeast, the maltodextrin from the liquefaction step must be further hydrolyzed in a saccharification step. The hydrolysis is preferably performed enzymatically using a carbohydrate-source generating enzyme, such as preferably glucoamylase. Alternatively, e.g., alpha-glucosidases, beta-amylase or acid alpha-amylases may be used. According to the present invention the saccharification and fermentation may be carried out simultaneously (SSF process).

The combined saccharification and fermentation process (SSF) may be carried out as defined above in the presence of a glucoamylase derived from a microorganism or a plant. Suitable carbohydrate-source generating enzymes, such as glucoamylases, include the one listed.

Carbohydrate-source generating enzymes and concentrations of use are described in the “Carbohydrate-Source Generating Enzyme”-section above.

Glucoamylases may in an embodiment be added in an amount of 0.02-2 AGU/g DS, preferably 0.1-1 AGU/g DS, such as 0.2 AGU/g DS. The ratio between acidic fungal alpha-amylase activity (AFAU) per glucoamylase activity (AGU) (AFAU per AGU) may in one embodiment be at least 0.1, preferably, at least 0.16, such as in the range from 0.12 to 0.30.

The fermenting organism is in one embodiment be a yeast, preferably one of the yeast mentioned in the Fermenting organisms” section above. The fermentation step may be in accordance with the fermentation process of the invention.

In ethanol production, the fermenting organism is preferably yeast, which is applied to the mash. Preferred yeast is derived from Saccharomyces spp., more preferably, from Saccharomyces cerevisiae. In preferred embodiments, yeast is applied to the mash and the fermentation is ongoing for 24-96 hours, such as typically 35-60 hours. In preferred embodiments, the temperature is generally between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

Distillation In a preferred embodiment the fermented mash is distilled using any method know in the art. The mash may be distilled to extract the fermentation product, in particular ethanol. The end product, obtained according to an ethanol production process of the invention may be used as, e.g., fuel ethanol; drinking ethanol, i.e., potable neutral spirits; or industrial ethanol.

Further details on how to carry out milling, liquefaction, saccharification, fermentation, distillation, and ethanol recovery are well known to the skilled person.

Materials and Methods

Glucoamylase SF: Balanced blend of Aspergillus niger glucoamylase and A. niger acid alpha-amylase having a ratio between AGU and AFAU of approx. 9:1.
   Yeast:
Red Star available from Red Star/Lesaffre, USA

Methods:

Alpha-amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.±0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| Substrate | Soluble starch | |
|---|---|---|
| Temperature | 37° | C. |
| pH | 4.7 | |
| Reaction time | 7-20 | minutes |

Determination of Acid Alpha-amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions. 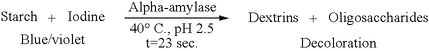
Standard conditions/reaction conditions: (per minute)
Substrate: Starch, approx. 0.17 g/L
Buffer: Citate, approx. 0.03 M
Iodine (I₂): 0.03 g/L
CaCl₂: 1.85 mM
pH: 2.50±0.05
Incubation temperature: 40° C.
Reaction time: 23 seconds
Wavelength: lambda=590nm
Enzyme concentration: 0.025 AFAU/mL
Enzyme working range: 0.01-0.04 AFAU/mL

If further details are preferred these can be found in EB-SM-0259.02/01 available on request from Novozymes A/S, Denmark, and incorporated by reference.

Acid Alpha-amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard Conditions/Reaction Conditions:

| | |
|---|---|
| Substrate: | Soluble starch. Concentration approx. 20 g DS/L. |
| Buffer: | Citrate, approx. 0.13 M, pH = 4.2 |
| Iodine solution: | 40.176 g potassium iodide + 0.088 g iodine/L |
| City water | 15°-20° dH (German degree hardness) |
| pH: | 4.2 |
| Incubation | 30° C. |
| temperature: | |
| Reaction time: | 11 minutes |
| Wavelength: | 620 nm |
| Enzyme | 0.13-0.19 AAU/mL |
| concentration: | |
| Enzyme working | 0.13-0.19 AAU/mL |
| range: | |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP 0140410, which disclosure is hereby incorporated by reference.

Glucoamylase Activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard Conditions/Reaction Conditions:
Substrate: Soluble starch.
Concentration approx. 16 9 dry matter/L.
Buffer: Acetate, approx. 0.04 M, pH=4.3
pH: 4.3
Incubation temperature: 60° C.
Reaction time: 15 minutes
Termination of the reaction: NaOH to a concentration of approximately 0.2 g/L (pH-9)
Enzyme concentration: 0.15-0.55 AAU/mL.

The starch should be Lintner starch, which is a thin-boiling starch used in the laboratory as calorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

Determination of Homology (Identity)

In context of the invention the term polypeptide “homology” is understood as the degree of “identity” between two sequences indicating a derivation of the first sequence from the second. The homology may suitably be determined by means of computer programs 10 known in the art such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-453. The following settings for amino acid sequence comparison are used: GAP creation penalty of 3.0 and GAP extension penalty of 0.1.

### EXAMPLES

### Example 1

Delayed Release of Glucose During SSF

The experiment was carried out in 500 ml shake flasks as triplicates. Enzyme 20 additions were dosed at different time points to examine the effect of a delayed release of glucose.

Two different experiments were carried out.

The first experiment investigated the impact from delaying the enzyme addition compared to the time of inoculation of yeast. Table 1 shows the dosing scheme of the experiment. Controls were included in the fermentation to have a reference yield from a standard fermentation with simultaneous yeast and enzyme addition.

TABLE 1
Delayed dosing of enzyme. Table shows time points of enzyme
addition and added amounts in percent of the total dose.

| Exp ID | Time [h] | | | Total |
|---|---|---|---|---|
| # | 0 | 8 | 24 | Dose |
| I | 100% | 0% | 0% | 100% |
| II | 0% | 100% | 0% | 100% |
| III | 0% | 0% | 100% | 100% |

The second experiment looked at distributing the enzyme dose addition over 24 hours. A small percentage of the total dose was added up front to provide low levels of available substrate in the beginning of the fermentation where the yeast cells are in the lag phase. Table 2 shows the dosing scheme where the initial addition ranges from 1%-20% of the total amount of enzyme added. Also in this experiment, controls were included for a 10 reference yield from a standard fermentation with simultaneous yeast and enzyme addition.

TABLE 2
Gradual Enzyme dosing. Time points and distribution
of enzyme dose in percentage of total added.

| Exp ID | Time [h] | | | Total |
|---|---|---|---|---|
| # | 0 | 8 | 24 | Dose |
| A | 0% | 0% | 100% | 100% |
| B | 1% | 0% | 99% | 100% |
| C | 5% | 0% | 95% | 100% |
| D | 10% | 0% | 90% | 100% |
| E | 20% | 0% | 80% | 100% |

TABLE 3
Loading Chart in % w/w-DS for the two conducted shake
flask experiments using Glucoamylase SF (GASF)

| | | | 0 h | 8 h | 24 h |
|---|---|---|---|---|---|
| | | | Glucoamyl- | Glucoamyl- | Glucoamyl- |
| | Exp ID | | ase SF | ase SF | ase SF |
| Exp 1 | I | 100 0 0 | 0.056% | — | — |
| | II | 0 100 0 | — | 0.056% | — |
| | III | 0 0 100 | — | — | 0.056% |
| Exp 2 | A | 100 0 0 | 0.056% | — | — |
| | B | 1 0 99 | 0.001% | — | 0.055% |
| | C | 5 0 95 | 0.003% | — | 0.053% |
| | D | 10 0 90 | 0.006% | — | 0.050% |
| | E | 20 0 80 | 0.011% | — | 0.045% |

The addition of Glucoamylase SF (GASF) was added as a total dose of 0.056% w-enzyme/w-DS_{Corn Mash}. Table 3 shows the amounts added in the two experiments at time 0, 8 and 24 hours.

Before transferring approximately 200 9 of corn mash to the 500 ml test bottles, the pH was recorded and penicillin (3 mg/kg), Urea (1 g/kg), and 4% w/w yeast propagate (overnight culture inoculated from freeze dried yeast cells, RedStar) were added. The enzyme was then added to the bottles according to the pre-determined loading (Table 3) and based on the mash-weight and the determined total solids (TS) content. A Denver DS-IR oven was used to determine the TS-value used for the enzyme addition calculations. The TS was subsequently determined as triplicate measurements after 24 hours drying at 105° C. The bottles were sealed with rubber stoppers and disposable pipettes having a single needle hole to release over pressure. Bottles were placed in 32° C. water bath with stirring at 100 rpm and the experiment start time was recorded. Enzyme additions during the fermentation were transferred to the bottles using pipettes. The rubber stopper was loosened and the enzyme added through a crack as small as possible (in order to maintain anaerobic conditions in the bottles). At 65 hours and 135 hours samples were taken for HPLC measurements.

Results

Experiment 1: The experiment where the GASF dose was delayed 8 and 24 hours respectively showed that it is possible to control the glycerol-ethanol relationship. From the results in Table 4 it can be seen that the late enzyme addition lowers the relative glycerol production compared to a standard SSF fermentation scheme. Furthermore, the results indicate that a total fermentation time of 65 hours is too short if the GASF is added 24 hours after yeast inoculation. The glycerol/ethanol relationship was lowered from 9% to 8.3%. In comparison most corn based ethanol plants have an approximate glycerol/ethanol relationship of 10% (w/w).

Experiment 2: In the second experiment where a fraction of the enzyme dose was added at the beginning of the experiment it was also seen that the dosing could effect the relative glycerol formation. As seen on Table 5 the lowest relative glycerol production was observed in the Batch A where all the GASF were added after 24 hours. The glycerol-ethanol relationship was found to increase with increased amounts of enzyme added in the beginning of the experiment. From Batch A to Batch E (Table 5) the difference in relative glycerol formation was 10%. When using liquefied corn mash it is expected that accessible sugars is already present before GASF is added, hereby having an initial level of substrate to secure yeast growth.

TABLE 4
Time point comparison, EXP1

| | | | | | Gly/EtOH | |
|---|---|---|---|---|---|---|
| Treatment | Glycerol [g/l] | | Ethanol [g/l] | | Relationship [% w/w] | |
| Exp 1 | 65 h | 135 h | 65 h | 135 h | 65 h | 135 h |
| I (100 0 0) | 12.3 | 12.5 | 138.0 | 138.1 | 8.9% | 9.0% |
| II (0 100 0) | 11.7 | 11.8 | 137.8 | 138.6 | 8.5% | 8.5% |
| III (0 0 100) | 11.5 | 11.5 | 136.8 | 138.5 | 8.4% | 8.3% |

TABLE 5
Time point comparison, EXP2

| | | | | | Gly/EtOH | |
|---|---|---|---|---|---|---|
| Treatment | Glycerol [g/l] | | Ethanol [g/l] | | Relationship [% w/w] | |
| Exp 2 | 65 h | 135 h | 65 h | 135 h | 65 h | 135 h |
| A (0 0 100) | 11.5 | 11.5 | 136.8 | 138.5 | 8.4% | 8.3% |
| B (1 0 99) | 11.7 | 11.8 | 137.3 | 137.4 | 8.5% | 8.6% |
| C (5 0 95) | 11.6 | 11.8 | 136.3 | 136.9 | 8.5% | 8.6% |
| D (10 0 90) | 12.2 | 12.4 | 135.5 | 135.0 | 9.0% | 9.2% |
| E (20 0 80) | 12.6 | 12.8 | 136.6 | 137.5 | 9.2% | 9.3% |

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. A process of producing a fermentation product in a fermentation medium which process comprises a fermentation step including subjecting liquefied mash to a carbohydrate-source generating enzyme and a fermenting microorganism, wherein the process comprises:
(a) introducing the fermenting organism into the fermentation medium,
(b) adding said carbohydrate generating enzyme after the lag phase of the fermenting organism,
(c) fermenting under conditions suitable for producing the fermentation product.

2. The process of claim 1, wherein the carbohydrate-source generating enzyme is added when the exponential growth phase of the fermenting organism is initiated.

3. The process of claim 1, wherein said fermenting organism is yeast.

4. The process of claim 1, wherein the carbohydrate-source generating enzyme is enzyme is glucoamylase or an alpha-amylase, or mixtures thereof, preferably in mixture of acidic fungal alpha-amylase activity (AFAU) per glucoamylase activity (AGU) (AFAU per AGU) of at least 0.1, in particular at least 0.16, such as in the range from 0.12 to 0.50.

5. The process of claim 1, wherein said fermentation product is ethanol.

6. The process of claim 1, wherein said fermentation step is part of a simultaneous saccharification and fermentation process.

7. A process for producing a fermentation product, comprising
(a) milling whole grains;
(b) liquefying the product of step (a);
(c) introducing the fermenting organism into the liquefied product obtained in step (b),
(d) adding the carbohydrate-source generating enzyme after the lag phase of the fermenting organism, and
(e) fermenting under conditions suitable for producing the desired fermentation product.

8. The process of claim 7, wherein the carbohydrate-source generating enzyme is added when the exponential growth phase of the fermenting organism is initiated.

9. The process of claim 7, further comprising distilling the fermented material.

10. The process of claim 7, wherein the carbohydrate-source generating enzyme is enzyme is glucoamylase or an alpha-amylase, or mixtures thereof, preferably in mixture of acidic fungal alpha-amylase activity (AFAU) per glucoamylase activity (AGU) (AFAU per AGU) of at least 0.1, in particular at least 0.16, such as in the range from 0.12 to 0.50.

11. The process of claim 7, wherein said fermenting organism is yeast.

12. The process of claim 7, wherein the fermentation product is ethanol.
